(19) United States
(12) Patent Application Publication
(10) Pub. No.: US 2007/0123988 A1
(43) Pub. Date: May 31, 2007

(54) Tapered anchor for tendon graft

(75) Inventor: Douglas M. Coughlin, Pinellas Park, FL (US)

Correspondence Address: HOLLAND & KNIGHT LLP;ATTN: STEFAN V. STEIN/ IP DEPT., POST OFFICE BOX 1288, TAMPA, FL, 33601-1288, US

(73) Assignee: Halkey-Roberts Corporation

(21) Appl. No.: 11/589,336

(22) Filed: Oct. 30, 2006

Related U.S. Application Data
(60) Provisional application No. 60/731,333, filed on Oct. 28, 2005.

(51) Int. Cl. A61F 2/44

(52) U.S. Cl. 623/17.14

(57) ABSTRACT

A tapered tendon graft anchor employed during a tendon graft reconstruction surgical procedure. The anchor of the invention comprises a “hair-pin” configuration formed of a loop and two opposing leafs. Each of the leafs include inwardly extending teeth in facing alignment with each other. The outer surfaces of the leafs are tapered to a frustro-conical shape. A harvested tendon with a bone block at one end is positioned with a cavity defined by the loop of the “hair pin” configuration with the tendon positioned between the inwardly extending teeth of the leafs. When the anchor is positioned within a hole in the bone of the joint whose ligament is to be reconstructed, the tendon is tightly grasped by the anchor to securely anchor the tendon in the hole allowing the bone block to be grafted within the hole.

### CROSS REFERENCE TO RELATED APPLICATIONS

This application claims the benefit of provisional patent application 60/731,333 filed Oct. 28, 2005, the disclosure of which is hereby incorporated by reference herein.

### BACKGROUND OF THE INVENTION

1. Field of the Invention

This invention relates to fixation devices that secure a bone tendon bone graft inside bone tunnel. More particularly, this invention relates to surgical devices for attaching a replacement anterior cruciate ligament (ACL) or other tendon/ligament, in a bone.

2. Description of the Background Art

Presently there exist many surgical techniques employed for replacing, reconstructing or securing synthetic or biological connective tissues to bone surfaces, such as attaching and maintaining an anterior cruciate ligament (ACL) within a knee. More recent surgical procedures for tendon replacement and reconstruction involve auto-grafting a tendon to the site of a torn or dislocated tendon. Early surgical procedures involved stapling the auto-grafted tendon into placement.

More particularly, reconstruction is the standard of care after an ACL injury. In surgery it is generally known to use an autograft taken e.g., from the knee of the patient, to replace the ruptured ACL. The two most commonly used are the bone-patellar tendon bone (BPTB) and the hamstring tendon (semitendinosus tendon with or without gracilis tendon). Allografts, synthetic grafts and quadriceps tendon grafts have also been used as ACL substitutes. The surgical techniques of the ACL reconstruction using bone-tendon bone (BTB) graft and hamstring tendon graft are described in detail in the following references: Beck, C. L., Jr.; Paulos, L. E.; Rosenberg, T. D.: “Anterior cruciate ligament reconstruction with the endoscopic technique,” Operative Techniques in Orthopaedics, 2:96-98, 1992; Stahelin, A. C.; Weiler, A.: “All-inside anterior cruciate ligament reconstruction using semitendinosus tendon and soft threaded biodegradable interference screw fixation,” Arthroscopy, 13:773-779, 1997; Fu, F. H.; Ma, C. B.: Anterior Cruciate Ligament Reconstruction Using Quadruple Hamstring. Operative Techniques in Orthopaedics, 9:264-272, 1999. Additional references of interest include Hoffman, R. F. G.; Peine, R; Bail, H. J.; Sudkamp, N. P.; Weiler, A.: “Initial fixation strength of modified patellar tendon grafts for anatomic fixation in anterior cruciate ligament reconstruction,” Arthroscopy, 15:392-399, 1999. The disclosures of each of the above references are hereby incorporated by reference herein.

More specifically, among the currently available soft tissue (hamstring) graft fixation implants, currently the most commonly used method to secure an ACL substitute to a bony drill-hole in an ACL reconstruction is the interference technique. In the interference technique, an interference screw is inserted into the space between the drill-hole and the end of the graft to lock the graft into the drill-hole. The fixation screws, like interference screws, are normally made of metal, like stainless steel or titanium or of a bioabsorbable polymer, like polylactide. Unfortunately, during interference screw insertion, technical complications sometimes are presented. The threads of the screw may damage the graft or the passing sutures, the graft may rotate with the screw so that the optimal position of the graft is lost, the graft may be damaged or the screw may be inserted non parallel (divergent) to the graft thereby significantly decreasing the strength of fixation.

There are also concerns specific to the metal interference screws. For example, in case of a need for revision surgery, metal screws can significantly complicate the operation, as the hardware inserted in the primary reconstruction may need to be removed, sometimes resulting in considerable loss of bone in the fixation site, and thus, decreasing the strength of the fixation of the revised graft. Additionally, metal screws have also been shown to disturb postoperative MRI evaluation.

The problems specific to metal screws can naturally be avoided by the use of screws made of bioabsorbable materials. However, other problems may arise, such as the bioabsorbable screw breaking during screw insertion. Also, the drill-hole usually has to be threaded for the insertion of the bioabsorbable screw. This delays the surgical operation and removes mechanically stronger cortical bone, thus reducing the grip of the screw into the bone. Trauma is also increased.

Several attempts have been done to develop other types of anchoring devices for fixation of tendons in ACL reconstruction to overcome the complications noted above when employing interference screws. More particularly, extra-articular or suspensory fixation methods have been developed that permit fixation outside the bone tunnel. Common techniques for external fixation include using staples placed either on the anterior cortex of the tibia or implants with staples that are placed partially within an entrance opening of the tunnel. Although these methods have been shown to provide superior fixation strengths to the interference technique, they require that a portion of the implant be exterior to the drill-hole.

Consistent with the surgical procedures noted above, many medical devices have been patented that facilitate the attachment of the auto-grafted tendon in a hole in a bone in a manner which minimizes the number of incisions and openings to the site that otherwise would be necessary. Representative medical devices are taught in the following United States patents, the disclosures of which are hereby incorporated by reference herein:

| | |
|---|---|
| Re. 34,871 | Process of Endosteal Fixation of a Ligament |
| 3,973,277 | Attaching Fibrous Connective Tissue to Bone |
| 5,234,430 | Orthopedic Fixation Screw and Method |
| 5,397,356 | Pin for Securing a Replacement Ligament to a Bone |
| 5,931,840 | Bone Fixator for A Ligament System |
| 5,961,520 | Endosteal Anchoring Device for Urging a Ligament |
| | Against a Bone Surface |
| 6,379,361 | Endosteal Anchoring Device for Urging a Ligament |
| | Against a Bone Surface |

For example, as taught by U.S. Pat. No. 5,397,356, one technique for securing a replacement tendon to a bone involves harvesting a tendon having a bony section or plug at one or both ends. The tendon is threaded into a drilled hole by a guide pin or K-wire and then the bone plug is secured into position by a specially-adapted threaded pin. Importantly, the threaded pin securing the replacement tendon engages through its bony plug to secure it into position within the hole whereupon, over time, the bony plug is grafted into the knee, thereby permanently securing the replacement tendon into position. Unfortunately, however, the threaded pin that secures the replacement tendon in the tunnel of the receptor bone is intended to be removed once the bone plug once the bone plug has become grafted.

Therefore, it is an object of this invention to provide an improvement which overcomes the aforementioned inadequacies of the prior art devices and provides an improvement which is a significant contribution to the advancement of the tendon graft anchor art.

Another object of this invention is to provide an anchor or implant for use in securing a transplant, such as a tendon graft, in a bone, such as a tibia, that would be bioabsorbable.

Another object of this invention is to provide an anchor or implant for use in securing a transplant, such as a tendon graft, in a bone, such as a tibia, that would provide a rigid fixation of the transplant at the tunnel opening (apertural/anatomic fixation).

Another object of this invention is to provide an anchor or implant for use in securing a transplant, such as a tendon graft, in a bone, such as a tibia, that would have no external hardware such that the implant would optimally be a completely intraboreal or endosteal design with no part of the implant protruding outside the bone drill-hole or be located on the outer surface of the tibia.

Another object of this invention is to provide an anchor or implant for use in securing a transplant, such as a tendon graft, in a bone, such as a tibia, that would provide circumferential contact, preferably 360 degree contact, between the transplant and bone drill-hole walls.

Another object of this invention is to provide an anchor or implant for use in securing a transplant, such as a tendon graft, in a bone, such as a tibia, that would enable tensioning of the transplant before it is secured into the drill-hole such as by pulling the transplant/implant construct by hand.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purposes of summarizing the invention, the invention comprises a tapered tendon graft anchor employed during a tendon graft surgical procedure. The anchor of the invention comprises a “hair-pin” configuration formed of a loop and two opposing leafs. Each of the leafs include inwardly extending barbs in facing alignment with each other. The outer surfaces of the leafs are tapered to a frustro-conical shape. The loop of the “hair pin” configuration define a cavity. An access hole is formed through the wall of the loop of the anchor.

Without departing from the spirit and scope of this invention, the anchor of the invention may be used in various tendon graft reconstructions. Some possible reconstructions include Achilles tendon reconstructions, reconstructions of surface tendons and anterior cruciate ligament (ACL) reconstructions. However, or the purpose of summarizing the invention and without limiting the use of the anchor of the invention, the anchor is described as used in connection with an ACL reconstruction.

During the ACL surgical procedure, a central strip of tendon graft of a desired length is outlined and removed along with an attached segment of bone (called “bone blocks”) at both ends from a suitable donor site of the patient or from a cadaver. Typically the tendon graft is harvested from the patella above and the tibia below of the patient's or cadaver's knee. The harvested tendon graft will be installed in tunnels in the tibia and the femur to function as a replacement for the ruptured ACL.

More particularly, a tapered tunnel is drilled through the tibia. The tapered tunnel is preferably of the same frustro-conical shape as the outer surfaces of the leafs of the anchor. The drilling (preferably untapered) then continues into the femur to form a hole that exits the top of the femur.

Holes are carefully drilled into both of the bone blocks to allow for suturing as hereinafter described. One of the bone blocks is carefully cut and shaped to fit into the cavity in the anchor of the invention (hereinafter referred to as the tibia bone block) whereas the other bone block (hereinafter referred to as the femur bone block) is carefully cut and shaped to fit into the hole in the femur.

During installation, long lengths of sutures (usually two) are threaded through each of the bone blocks. The sutures of the tibia bone block are threaded through the hole in the loop of anchor of the invention and pull through until the tibia bone block is positioned within the cavity of the anchor. The harvested tendon is positioned between the leafs of the anchor along the length thereof.

A standard K-guide wire is connected to the sutures of the femur bone block. The K-guide wire is then threaded through the tapered tunnel in the tibia, then through the hole of the femur to exit the top of the femur. The exiting sutures may then be pulled until the femur bone block and the anchor of the invention are pulled into the hole and the tapered hole, respectively.

It is noted that due to the matching frustro-conical shapes of the anchor and tapered hole, the inwardly facing teeth of the leafs progressively grasp the tendon positioned between the leafs of the anchor as the anchor of the invention is progressively pulled into the tapered hole. Once the anchor is fully seated within the tapered hole in the tibia, the tendon is securing retained in position without the need for any other fastener such as a locking or interference screw.

Once the tendon is tensioned by pulling on the sutures existing the top of the femur, the femur bone block may be secured into position within the femoral hole by any conventional method such as by employing an interference screw and/or by using an external tensioning screw as known as the ™“Endo Button” sold by Linvatec. Irrespective of the method employed for tensioning, it is noted that the more the tendon is tensioned to achieve the desired tendon load in the joint, the more the anchor of the invention is wedged tightly into the tapered tunnel in the tibia to more securely grasp the portion of the tendon positioned between the leafs of the anchor. Further, it is noted that the engagement of the anchor within the tapered tunnel prevents the tendon and the femur bone block from moving after being positioned inside the tunnel.

The anchor of the invention is preferably composed of a bioresorbable material. Representative types of bioresorbable materials include, without limitation, the materials known as ™“PolyGraft” sold by OsteoBiologics, Inc., ™“Osteo-Pin” [Ploy (L-lactide-co-D,L-lactide) 70/30] sold by OsteoHealth Company or ™“LactoSorb” sold by Lorenz Surgical, Inc. Being composed of a bioresorbable material, the anchor of the invention will over time be resorbed into the bone as the tibia bone block is grafted into the tapered hole.

The anchor of the invention is located totally inside of the tapered hole in the tibia so that no part of it protrudes from the outer surface of the bone. Therefore, it provides an ACL reconstruction fixation apparatus and method that does not damage the tendon graft and minimally disturbs the tissue metabolism and blood circulation while securely grasps the tendon graft inside of the tapered hole, thus enabling maximum filling of the hole with the tendon and facilitating effective re-growth of bone to tendon within tapered hole. Advantageously, the preferred embodiment of the anchor of the invention is bioresorbable and results in a strong and rigid fixation of the tendon graft in ACL reconstruction that does not interfere with non-invasive examinations such as radiographs, MRI or CT.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings in which:

FIG. 1 is a perspective view of the anchor of the invention showing the “hairpin” configuration comprised of opposing leafs interconnected by a loop;

FIG. 2 is side plan view of FIG. 1 showing the inwardly-facing staggered teeth of the opposing leafs of the anchor;

FIG. 3 is a side plan view of the anchor of the invention with a harvested tendon graft installed between the opposing leafs to be grasped by the inwardly-facing staggered teeth thereof and with the bone block at the end of the harvested tendon graft positioned within the cavity formed in the loop of the anchor;

FIG. 4 is a cross-sectional view of the anchor with a harvested tendon graft installed within a tapered hole in a bone;

FIG. 5 is a side view of a knee, partially in cross-section, showing the manner in which the anchor of the invention and the harvested tendon is used in connection with an ACL reconstruction; and

FIG. 6 is another side view of a knee, partially in cross-section, showing the use of an interference screw to secure the femur bone block within the femur once the tendon is tensioned.

Similar reference characters refer to similar parts throughout the several views of the drawings.

### DETAILED DESCRIPTION OF THE PREFERRED EMBODIMENT

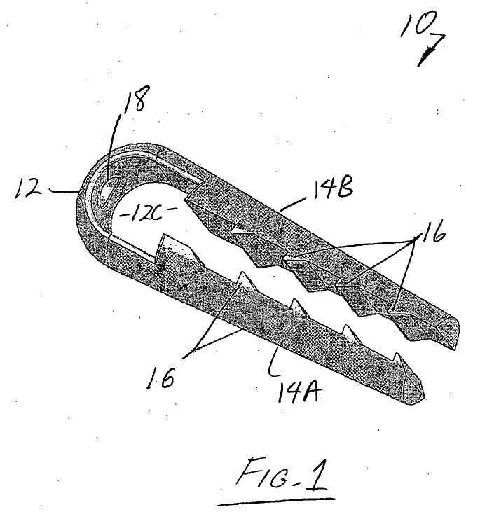

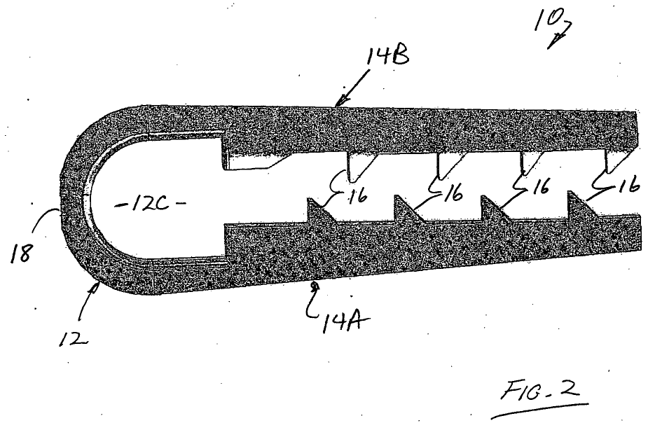

Referring to FIGS. 1 and 2, the anchor 10 of the invention comprises a loop 12 having two opposing leafs 14A and 14B formed in a “hairpin” configuration. The loop 12 defines a interior cavity 12C and functions as a living hinge to allow the opposing leafs 14A and 14B to flex inwardly toward each other. The outer surfaces of the opposing leafs 14A and 14B are frustro-conically shaped. The inner surfaces of the opposing leafs 14A and 14B comprise inwardly extending teeth 16 along the length thereof in facing alignment with each other. The leaves 14A and 14B define a longitudinal path therebetween from the cavity 12C. An access hole 18 is formed through the loop 12 of the anchor 10 to allow suture access into the cavity 12C (as described hereinafter). Preferably, as best shown in FIG. 2, the inwardly disposed teeth 16 along one leaf 14A are staggered relative to the opposing teeth 16 of the other leaf 14B.

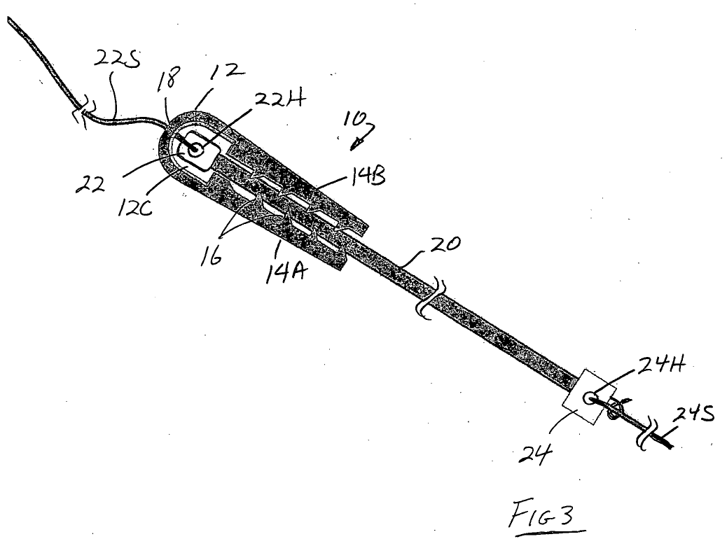

Referring to FIG. 3, the anchor 10 of the invention is loaded with a harvested grafted tendon 20, preferably having a bone block 22 on one end thereof. The grafted tendon 20 may be also harvested with a bone block 24 at its other end. A holes 22H and 24H is drilled through the respective bone blocks 22 and 24. At least one suture 22S and 24S is then threaded through each of the respective holes 22H and 24H and tightly tied to the respective bone block 22S and 24S.

The anchor 10 is loaded with the harvested tendon 20 by threading the end of the suture 22S through the access hole 18 and pulling on it until the first bone block 22 is positioned within the cavity 12C formed by the loop 12 of the anchor 10. The tendon 20 is then positioned between the opposing leafs 14A and B along their longitudinal lengths to be engaged by the inwardly-disposed staggered teeth 16 thereof.

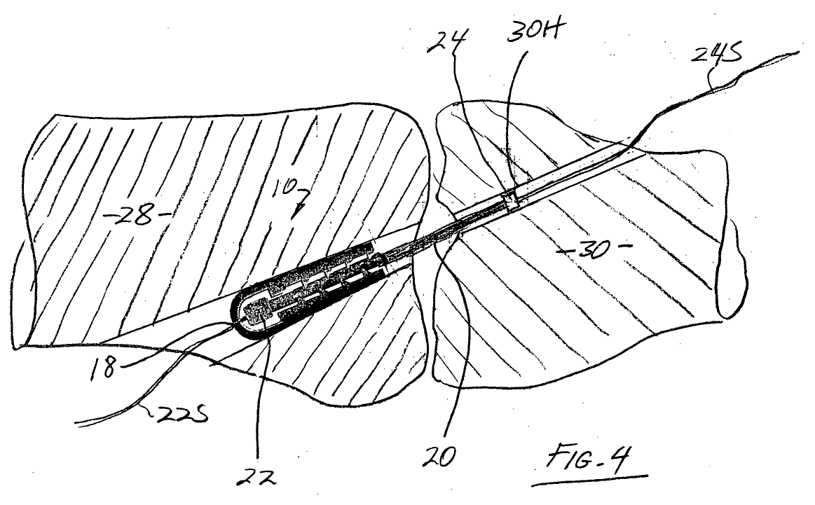

Referring now to FIG. 4, the anchor 10 of the invention with the one end of the grafted tendon 20 installed therein is fitted into a frustro-conical tapered hole or tunnel 28H drilled into a first bone 28 whereupon the other end of the grafted tendon 20 is then grafted to the complementary second bone 30 to reconstruct the ligamented joint. The installation of the anchor 10 of the invention in the first bone preferably occurs by threading the suture 24S through the tapered hole 28H (optionally with the assistance of a K-guide wire connected to its end). The suture 24S may then be pulled to force the anchor 10 into the tapered hole 28H. It is noted that a slight pinching of the anchor 10 may be necessary to start it into the tapered hole 28H in the first bone 28. As the suture 24S is pulled under tension, the anchor 10 more fully enters the tapered hole 26 whereupon the inwardly-disposed staggered teeth 16 of the opposing leafs 14A and 14B more tightly grasps the tendon 20 therebetween due to the matching frustro-conical shape of the outer surfaces of the opposing leafs 14 and the frustro-shape of the tapered hole 28H. The staggered arrangement of the teeth 16 assure that the tendon 20 will be tightly gripped without damage that might otherwise occur if the row of teeth 16 in each leaf 14A and 14B were in opposing alignment with each other.

The other end of the grafted tendon 20 may then be secured to a second bone 30 whose ligamented joint is to be reconstructed by using any conventional technique. One method may comprise threading the suture 24S through a hole 30H drilled in the second bone 30 to exit the second bone 30 to pull the second bone block 30 into the hole 30H and tightly tensioning the tendon 20, whereupon the second bone block 30 may then be secured into position within the hole 30H by tying off the suture external to the second bone (e.g., by using an ™“Endo Button”), by using an interference screw or by using any other available technique or medical device.

It is noted that the sutures 22S and 24S tied to the respective bone blocks 22 and 24 allow the tendon 20 with its bone blocks 22 and 24 to be alternatively pulled back and forth during installation within the respective holes 22H and 24H to make sure they are properly fitted therein. It is also noted that the diameter and shape of the hole 30H is preferably substantially the same as that of the second bone block 24 to allow the second bone block 24 to slide therein and be grafted to the second bone 20.

Preferably, the anchor 10 of the invention is composed of a bioresorbable material that is absorbed into the first bone 28 over time as the bone block 22 is grafted. Until such resorbing and grafting occurs, however, it is emphasized that the inwardly-facing staggered teeth 16 of the anchor 10 firmly grasp the tendon 20 to preclude any slippage.

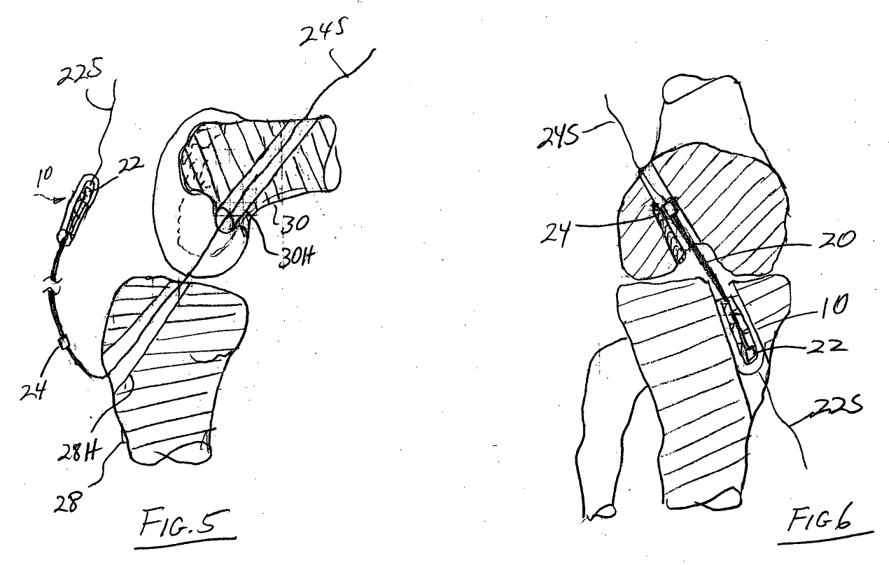

The foregoing has described the anchor 10 of the invention in a general sense applicable to many different ligament reconstructions. With limitation on the use of the anchor 10 of the invention, the anchor 10 is particularly suitable for use in an ACL reconstruction. More particularly, as shown in FIGS. 5 and 6, the first bone 28 may comprise a tibia 28 and the second bone 30 may comprise the femur 30. The tapered hole 28H is formed through the tibia 28 whereas the hole 30H is formed through the femur 30. The harvested graft tendon 20 is installed within the anchor 10 (in the manner shown in FIG. 3) and then the assembly is threaded the holes 28H, 30H and 30R of the tibia 28 and the femur to force the anchor 10 into the tapered hole 28H in the tibia 28 with the suture 24S exiting the hole 30H on the top of the tibia 30 (see FIG. 5). Upon tensioning of the tendon 20 by pulling on the suture 24S exiting the hole 30H, an interference screw 32 may be installed from the underside of the tibia 30 to secure the second bone block 24 within the hole 30H.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. an apparatus for securing a grafted tendon within a hole in a bone, comprising in combination:
a tendon;
an anchor, said anchor including:
a loop; and
at least two opposing leafs capable of flexing toward one another;
said anchor being positioned within the hole in the bone; and
said tendon being positioned between said leafs to be grasped thereby as they flex toward one another when said anchor is positioned with a hole in a bone.

2. The apparatus as set forth in claim 1, wherein at least one inwardly extending tooth is formed along an inside surface of at least one of said leafs.

3. The apparatus as set forth in claim 2, further including:
a cavity formed in said loop;
said tendon further including a bone block that is positioned within said cavity.

4. The apparatus as set forth in claim 3, wherein a suture is connected to said bone block and wherein said loop further includes an access hole to allow said suture to be threaded therethrough.

5. The apparatus as set forth in claim 2, further comprising a plurality of said teeth positioned on both said leafs.

6. The apparatus as set forth in claim 5, wherein said teeth along one said leafs are staggered relative to opposing said teeth of the other said leaf.

7. The apparatus as set forth in claim 1, wherein an outer surface of at least one of said leafs of said anchor comprises a taper.

8. The apparatus as set forth in claim 7, wherein said outer surfaces of both said leaf comprise a taper to define a frustro-conical configuration.

9. The apparatus as set forth in claim 7, wherein said hole in said bone is tapered.

10. The apparatus as set forth in claim 8, wherein said hole in said bone is tapered.

11. The apparatus as set forth in claim 1, wherein said anchor is formed of a bioresorbable material.

12. The apparatus as set forth in claim 1 wherein said bone comprises a tibia and wherein said tendon comprises a replacement ACL tendon.

13. A surgical method employing the apparatus as set forth in claim 1.

14. A surgical method comprising the steps of:
harvesting a tendon;
drilling a hole in a bone;
positioning the tendon in an anchor, said anchor including a loop and at least two opposing leafs capable of flexing toward one another about said tendon positioned therebetween;
positioning said anchor within the hole in the bone; and
said leafs grasping said tendon as the anchor is forced within said hole in the bone.

15. The method as set forth in claim 14, wherein at least one inwardly extending tooth is formed along an inside surface of at least one of said leafs.

16. The method as set forth in claim 15, wherein the step of harvesting said tendon comprises harvesting said tendon with a bone block and wherein said anchor comprises a cavity formed in said loop into which said bond block is positioned.

17. The method as set forth in claim 16, further comprising the step of connecting a suture to said bone block which is then threaded through an access hole formed in said loop.

18. The method as set forth in claim 17, wherein each of said leafs comprise a plurality of inwardly said teeth that are staggered relative to opposing said teeth of the other said leaf.

19. The method as set forth in claim 18, wherein an outer surface of at least one of said leafs of said anchor comprises a taper and wherein said hole comprises a tapered hole.

20. The method as set forth in claim 19, wherein said outer surfaces of both said leaf comprise a taper to define a frustro-conical configuration.

21. The method as set forth in claim 20, wherein said anchor is formed of a bioresorbable material.

22. The method as set forth in claim 21, wherein said bone comprises a tibia and wherein said tendon comprises a replacement ACL tendon.
